(19) United States
(12) Patent Application Publication
(10) Pub. No.: US 2010/0003190 A1
(43) Pub. Date: Jan. 7, 2010

(54) Method for protecting renal tubular epithelial cells from radiocontrast nephropathy (RCN)

(75) Inventors: Alexey Y. Kolyada, Newton, MA (US); Bertrand L. Jaber, South Boston, MA (US); Orfeas Liangos, Brighton, MA (US); Nicolaos E. Madias, Newton, MA (US)

Correspondence Address: WOLF GREENFIELD & SACKS, P.C., 600 ATLANTIC AVENUE, BOSTON, MA, 02210-2206, US

(73) Assignee: Caritas St. Elizabeth's Medical Center of Boston, Inc., Boston, MA (US)

(21) Appl. No.: 12/312,932

(22) PCT Filed: Dec. 7, 2007
(86) PCT No.: PCT/US07/25066

§ 371 Date: Jun. 1, 2009

Related U.S. Application Data
(60) Provisional application No. 60/873,724, filed on Dec. 8, 2006.

(51) Int. Cl. A61K 51/00; C12Q 1/02

(52) U.S. Cl. 424/1.65; 435/29

(57) ABSTRACT

According to aspects of the invention, methods for preventing or treating kidney damage are provided. Compositions for the prevention or treatment of kidney damage are also provided. Aspects of the invention relate to methods for screening the toxicity of a radio-contrast agent. Kits for the treatment or prevention of kidney damage are also provided.

### RELATED APPLICATIONS

This application claims priority under 35 U.S.C. § 119(e) from U.S. provisional application Ser. No. 60/873,724 filed Dec. 8, 2006, the entire content of which is incorporated by reference herein.

### FIELD OF THE INVENTION

Aspects of the invention relate to methods and compositions for preventing or treating kidney damage by administering a tissue protective agent. Aspects of the invention relate to kits for the treatment or prevention of kidney damage. Aspects of the invention relate to methods for screening a tissue protective agent in vitro.

### BACKGROUND OF THE INVENTION

Iodinated radiographic contrast media are increasingly used as a result of ongoing progress in diagnostic imaging techniques. Use of such media, however, is not without risk. Radiocontrast nephropathy (RCN) refers to an acute transient impairment of renal function after the intravascular administration of radiocontrast medium (Nikolsky E, et al. Rev Cardiovasc Med, 2003, 4 Suppl 1:S7-S14). In patients with normal renal function, the frequency of RCN is low (5%), but could be as high as 50% in high-risk patients (Rudnick M R, et al., Kidney Int, 1995, 47(1):254-61; Morcos S K, et al., Eur Radiol., 1999, 9(8):1602-13; Rihal C S, et al., Circulation, 2002, 105(19):2259-64). The development of RCN is linked to increased cardiovascular complications, prolonged hospitalization and higher in hospital and long-term mortality (Rihal C S, et al., Circulation, 2002, 105(19):2259-64; Gruberg L, et al., J Am Coll Cardiol, 2000, 36(5): 1542-8; Freeman R V, et al., Am J Cardiol, 2002, 90(10):1068-73).

Several factors, including chronic kidney disease, diabetes mellitus, intravascular volume depletion and dose of radiocontrast media, have been shown to significantly increase the risk of RCN (Nikolsky E, et al. Rev Cardiovasc Med, 2003, 4 Suppl 1:S7-S14; Rihal C S, et al., Circulation, 2002, 105(19):2259-64; Gruberg L, et al., J Am Coll Cardiol, 2000, 36(5):1542-8; Morcos S K., Clin Radiol, 2004, 59(5):381-9; Rich M W, Crecelius C A., Arch Intern Med, 1990, 150(6):1237-42; McCullough P A, et al., Am J Med, 1997, 103(5):368-75). Because the occurrence of RCN may be predicted in most of these cases, preventive strategies represent a logical therapeutic approach. Accordingly, several studies have evaluated different pharmacological agents (furosemide, mannitol, dopamine, calcium channel blockers, endothelin receptor antagonists, theophylline, fenoldopam and prostaglandin) used prophylactically before radiocontrast media exposure. None of these strategies has conclusively shown a clear benefit (Morcos S K., Clin Radiol, 2004, 59(5):381-9; Cox C D, Tsikouris J P., J Clin Pharmacol, 2004, 44(4):327-37). Improved methods that reduce renal tubular epithelial cell damage induced by radiocontrast media, and as a result, might reduce the occurrence of RCN are therefore needed.

It has previously been shown that rhEpo is neuro-protective after brain ischemia-reperfusion injury (Sakanaka M, et al., Proc Natl Acad Sci USA, 1998, 95(8):4635-40; Brines M L, et al., Proc Natl Acad Sci USA, 2000, 97(19):10526-31); rhEpo is protective in the retina (Junk A K, et al., Proc Natl Acad Sci USA, 2002, 99(16):10659-64), in cardiac ischemia-reperfusion models (Calvillo L, et al., Proc Natl Acad Sci USA, 2003, 100(8):4802-6; Parsa C J, et al., J Biol Chem, 2004, 279(20):20655-62), spinal cord and peripheral nerve compression (Gorio A, et al., Proc Natl Acad Sci USA, 2002, 99(14):9450-5; Sekiguchi Y, et al., Spine, 2003, 28(23):2577-84), and skin wound healing models (Buemi M, et al., Acta Dermn Venereol, 2002, 82(6):411-7). In a rat cisplatinum kidney injury model, rhEpo enhanced the rate of renal recovery (Bagnis C, et al., Nephrol Dial Transplant, 2001, 16(5):932-8), and in a model of renal ischemia-reperfusion injury, it reduced tubular cells apoptosis (Vesey D A, et al., Nephrol Dial Transplant, 2004, 19(2):348-55; Johnson D W, et al., Kidney Int, 2006, 69(10):1806-13). It was not previously known that rhEpo protects against RCN.

Since pharmacological doses of rhEpo and darbepoetin facilitate their hematopoietic actions and, possibly, some unwanted effects, novel analogues of erythropoietin that are devoid of hematopoietic activity but are still tissue protective have been developed (Erbayraktar S, et al., Proc Natl Acad Sci USA, 2003, 100(11):6741-6; Leist M, et al., Science, 2004, 305(5681):239-42). Indeed, carbamylated rhEpo analogues are particularly intriguing because they do not bind to the classical erythropoietin receptor but still confer similar protection against experimental stroke, spinal cord injury and diabetic neuropathy as rhEpo, but are void of hematopoietic effects (Moon C, et al., J Pharmacol Exp Ther, 2006, 316(3):999-1005; Leist M, et al., Science, 2004, 305(5681):239-42).

The ability of rhEpo or analogues thereof to protect renal tubular cells against damage from radiocontrast media was not known.

### SUMMARY OF THE INVENTION

Aspects of the invention relate to methods for treating or preventing kidney damage. In one embodiment, methods for treating or preventing in a subject kidney damage associated with the administration of a radio-contrast agent are presented. In some embodiments, a tissue protective agent in an amount effective to treat or prevent radiocontrast induced kidney damage or radio-contrast nephropathy and a radio-contrast agent in an amount effective to perform a radiographical examination, are administered to a subject. In certain embodiments, the tissue protective agent is an erythropoiesis-stimulating agent. In some embodiments, the erythropoiesis-stimulating agent is erythropoietin, epoetin-alpha, epoetin-beta, epoetin-delta, epoetin-omega, darbepoetin, asialoerythropoietin, carbamylated erythropoietin, continuous erythropoietin receptor activator, HIF prolyl hydroxylase inhibitors, angelica sinensis polysaccharides, EPREX™, NeoRecormon (epoetin-beta), Dynepo, or a synthetic non-recombinant pegylated, peptidic erythropoiesis-stimulating agent. In other embodiments, the tissue protective agent is erythropoietin. In some embodiments, the radio-contrast agent is iohexol. In other embodiments, the radio-contrast agent is iodixanol. In certain embodiments, the tissue protective agent is darbepoietin. In some embodiments, the radio-contrast agent is iohexol. In other embodiments, the radio-contrast agent is iodixanol. In certain embodiments, the radio-contrast agent is Diatrizoate (amidotrizoic acid, or 3,5-Diacetamido-2,4,6-triiodobenzoic acid), Metrizoate (water-soluble, nephrotropic, high osmolar X-ray contrast media), Ioxaglate (water-soluble, nephrotropic, low osmolar X-ray contrast media), Iopamidol (water-soluble, nephrotropic, low osmolar X-ray contrast media), Iohexol, Iopromide (water-soluble, nephrotropic, low osmolar X-ray contrast media), or Iodixanol. In other embodiments, the radio-contrast agent is iohexol. In some embodiments, the radio-contrast agent is iodixanol.

In certain embodiments of the invention, the tissue protective agent and the radio-contrast agent are administered simultaneously. In some embodiments, the tissue protective agent is administered prior to the radio-contrast agent. In other embodiments, the tissue protective agent is administered after the radio-contrast agent. In certain embodiments, the tissue protective agent is administered to a subject having chronic kidney disease. In some embodiments, the tissue protective agent is administered to a subject having diabetes mellitus.

In aspects of the invention, compositions for the treatment or prevention of kidney damage in a subject associated with the administration of a radio-contrast agent are provided. In certain embodiments, a tissue protective agent in an amount effective to treat or prevent radio-contrast induced kidney damage or radio-contrast nephropathy, and a radio-contrast agent in an amount effective to perform a radiographical examination, are included in the composition. In some embodiments, the tissue protective agent is an erythropoiesis-stimulating agent. In other embodiments, the erythropoiesis-stimulating agent is erythropoietin, epoetin-alpha, epoetin-beta, epoetin-delta, epoetin-omega, darbepoetin, asialoerythropoietin, carbamylated erythropoietin, continuous erythropoietin receptor activator, HIF prolyl hydroxylase inhibitors, angelica sinensis polysaccharides, EPREX™, NeoRecormon (epoetin-beta), Dynepo, or a synthetic non-recombinant pegylated, peptidic erythropoiesis-stimulating agent. In certain embodiments, the tissue protective agent is erythropoietin. In some embodiments, the radio-contrast agent is iodixanol. In other embodiments, the radio-contrast agent is iohexol. In some embodiments, the tissue protective agent is darbepoetin. In other embodiments, the radio-contrast agent is iodixanol. In some embodiments, the radio-contrast agent is iohexol. In certain embodiments, the radio-contrast agent is Diatrizoate (amidotrizoic acid, or 3,5-Diacetamido-2,4,6-triiodobenzoic acid), Metrizoate (water-soluble, nephrotropic, high osmolar X-ray contrast media), Ioxaglate (water-soluble, nephrotropic, low osmolar X-ray contrast media), Iopamidol (water-soluble, nephrotropic, low osmolar X-ray contrast media), Iohexol, Iopromide (water-soluble, nephrotropic, low osmolar X-ray contrast media), or Iodixanol. In some embodiments, the radio-contrast agent is iodixanol. In other embodiments, the radio-contrast agent is iohexol.

According to aspects of the invention, kits including a package containing a tissue protective agent in an amount effective to treat or prevent radiocontrast induced kidney damage or radio-contrast nephropathy and a radio-contrast agent in an amount effective to perform a radiographical examination, and instructions for use are provided. In certain embodiments, the tissue protective agent is an erythropoiesis-stimulating agent. In some embodiments, the erythropoiesis-stimulating agent is erythropoietin, epoetin-alpha, epoetin-beta, epoetin-delta, epoetin-omega, darbepoetin, asialoerythropoietin, carbamylated erythropoietin, continuous erythropoietin receptor activator, HIF prolyl hydroxylase inhibitors, angelica sinensis polysaccharides, EPREX™, NeoRecormon (epoetin-beta), Dynepo, or a synthetic non-recombinant pegylated, peptidic erythropoiesis-stimulating agent. In other embodiments, the tissue protective agent is erythropoietin. In some embodiments, the radio-contrast agent is iodixanol. In other embodiments, the radio-contrast agent is iohexol. In certain embodiments, the tissue protective agent is darbepoetin. In other embodiments, the radio-contrast agent is iodixanol. In some embodiments, the radio-contrast agent is iohexol.

In certain embodiments of the invention, the radio-contrast agent is Diatrizoate (amidotrizoic acid, or 3,5-Diacetamido-2,4,6-triiodobenzoic acid), Metrizoate (water-soluble, nephrotropic, high osmolar X-ray contrast media), Ioxaglate (water-soluble, nephrotropic, low osmolar X-ray contrast media), Iopamidol (water-soluble, nephrotropic, low osmolar X-ray contrast media), Iohexol, Iopromide (water-soluble, nephrotropic, low osmolar X-ray contrast media), or Iodixanol. In some embodiments, the radio-contrast agent is iodixanol. In other embodiments, the radio-contrast agent is iohexol. In certain embodiments, the tissue protective agent and the radio-contrast agent are individual preparations. In other embodiments, the tissue protective agent and the radio-contrast agent is a combined preparation.

According to aspects of the invention, methods for screening in vitro are provided. In certain embodiments, methods include contacting a cell culture with a tissue protective agent and a radio-contrast agent, incubating the cell culture with the tissue protective agent and the radio-contrast agent, and determining the cell viability of the cell culture. In certain embodiments, the tissue protective agent is an erythropoiesis-stimulating agent. In other embodiments, the erythropoiesis-stimulating agent is erythropoietin, epoetin-alpha, epoetin-beta, epoetin-delta, epoetin-omega, darbepoetin, asialoerythropoietin, carbamylated erythropoietin, continuous erythropoietin receptor activator, HIF prolyl hydroxylase inhibitors, angelica sinensis polysaccharides, EPREX™, NeoRecormon (epoetin-beta), Dynepo, or a synthetic non-recombinant pegylated, peptidic erythropoiesis-stimulating agent.

In certain embodiments of the invention, the cells are human kidney cells. In some embodiments, the human kidney cells are human renal tubular epithelial HK-2 cells. In other embodiments, the radio-contrast agent is added at a concentration of 100 mg/ml. In certain embodiments, the radio-contrast agent is Diatrizoate (amidotrizoic acid, or 3,5-Diacetamido-2,4,6-triiodobenzoic acid), Metrizoate (water-soluble, nephrotropic, high osmolar X-ray contrast media), Ioxaglate (water-soluble, nephrotropic, low osmolar X-ray contrast media), Iopamidol (water-soluble, nephrotropic, low osmolar X-ray contrast media), Iohexol, Iopromide (water-soluble, nephrotropic, low osmolar X-ray contrast media), or Iodixanol. In some embodiments, cell viability is determined using a colorimetric assay. In other embodiments, the colorimetric assay is the MTT assay.

In certain embodiments of the invention, the tissue protective agent and the radio-contrast agent are added simultaneously. In other embodiments, the tissue protective agent is added prior to the radio-contrast agent. In some embodiments, the radio-contrast agent is added prior to the tissue protective agent.

### BRIEF DESCRIPTION OF THE DRAWINGS

FIG. 1. Graph of cell viability of HK-2 cells in the presence of serum-free DMEM media, diatrizoate (ionic, high-osmolar), iohexol (nonionic, low-osmolar), or iodixanol (nonionic, iso-osmolar). The data are presented as mean values±SEM (N=3).

FIG. 2. Graph of cell viability of HK-2 cells+iohexol in the presence of increasing concentrations of rhEpo (0-400 U/ml). The data are presented as mean values±SEM (N=6). P=0.017 (by Friedman test). *P=0.027 vs. iohexol alone (by Wilcoxon Rank test).

FIG. 3. Graph of cell viability of HK-2 cells. a) HK-2 cells+iohexol (100 mg iodine/ml) in the absence of presence of rhEpo (200 U/ml). The data are presented as mean values±SEM (N=6). *P=0.028 vs. iohexol alone (by Wilcoxon Rank test). b) HK-2 cells+iodixanol (100 mg iodine/ml) in the absence of presence of rhEpo (200 U/ml). The data are presented as mean values±SEM (N=6). *P=0.027 vs. iodixanol alone (by Wilcoxon Rank test).

FIG. 4. Graph of cell viability of LLC-PK1 cells. a) LLC-PK1 cells+iohexol (100 mg iodine/ml) in the absence or presence of rhEpo (200 U/ml). The data are presented as mean values±SEM (N=9). *P=0.008 vs. iohexol alone (by Wilcoxon Rank test). b) LLC-PK1 cells+iodixanol (100 mg iodine/ml) in the absence or presence of rhEpo (200 U/ml). The data are presented as mean values±SEM (N=6). *P=0.028 vs. iodixanol alone (by Wilcoxon Rank test).

FIG. 5. Graph of cell viability of LLC-PK1 cells+iohexol (100 mg iodine/ml) in the absence of presence of different concentrations of darbepoetin (0-120 ng/ml). The data are presented as mean values±SEM (N=15). P<0.0001 (by Friedman test). *P=0.001 vs. iohexol alone (by Wilcoxon Rank test).

FIG. 6. Graph of caspase 3 activity of LLC-PK1 cells in the presence of iohexol (100 mg iodine/ml) or iodixanol (100 mg iodine/ml), and a caspase 3 inhibitor (DEVD-CHO, 50 μM). The data are presented as mean values±SEM (N=8). P<0.0001 (by Friedman test). *P=0.017 vs. DMEM (by Wilcoxon Rank test). **P=0.012 vs. DMEM (by Wilcoxon Rank test).

FIG. 7. Graph of caspase 3 activity of LLC-PK1 cells in the presence of iohexol (100 mg iodine/ml) with or without rhepo (200 U/ml). The data are presented as mean values±SEM (N=6). P=0.008 (by Friedman test). *P=0.028 vs. DMEM (by Wilcoxon Rank test). **P=0.043 vs. iohexol (by Wilcoxon Rank test).

### DETAILED DESCRIPTION OF THE INVENTION

Recombinant human erythropoietin (rhEpo), a widely available agent for the treatment of anemia, was recently found to reduce injury caused by ischemia-reperfusion of the brain, retinal neurons and heart (Brines M, Cerami A., Nat Rev Neurosci, 2005, 6(6):484-94; Joyeux-Faure M, et al., Fundam Clin Pharmacol, 2005, 19(4):439-46). Endogenous erythropoietin is primarily produced by renal cortical fibroblasts, and is considered to provide important paracrine cytoprotective effects within the kidney (Kuriyama S, et al., Nephron, 1997, 77(2):176-85; Jungers P, et al., Nephrol Dial Transplant, 2001, 16(2):307-12), as functional erythropoietin receptors are expressed on renal tubular epithelial cells (Westenfelder C, et al., Kidney Int, 1999, 55(3):808-20). It was previously unknown that rhEpo could protect against kidney damage or injury associated with the administration of a radio-contrast agent.

According to an aspect of the invention, methods and compositions for preventing or treating kidney damage are provided. It was discovered, according to the invention, that the administration of tissue protective agents may prevent or reduce radiocontrast media-induced injury of the kidney. In other aspects of the invention, methods for screening the toxicity of a radio-contrast agent in the presence of a tissue protective agent in vitro are also provided. Kits for the treatment or prevention of kidney damage are also provided.

According to aspects of the invention, methods for the treatment or prevention of kidney damage are provided. In some aspects, kidney damage may be a result of the administration of iodinated radiographic contrast media. In certain cases the kidney damage is a result of the intravascular administration of iodinated contrast materials during radiographical examination. The administration of such radio-contrast agent can cause radio-contrast nephropathy (RCN), an acute transient impairment of renal function.

A radio-contrast agent is a compound that is used to improve the visibility of internal bodily structures in an X-ray image or in Magnetic Resonance Imaging (MRI). A radio-contrast agent may be ionic or nonionic. A radio-contrast agent may be an iodinated radio-contrast agent. Iodinated radio-contrast agents include, but are not limited to, Diatrizoate (amidotrizoic acid, or 3,5-Diacetamido-2,4,6-triiodobenzoic acid, Hypaque® 50, Nycomed Imaging), Metrizoate (Isopaque® Coronar 370, water-soluble, nephrotropic, high osmolar X-ray contrast media), Ioxaglate (Hexabrix®, water-soluble, nephrotropic, low osmolar X-ray contrast media), Iopamidol (Isovue® 370, Water-soluble, nephrotropic, low osmolar X-ray contrast media), Iohexol (Omnipaque®, a contrast agent generally used during coronary angiography), Iopromide (water-soluble, nephrotropic, low osmolar X-ray contrast media), and Iodixanol (Visipaque 320®, contrast agent generally used during coronary angiography).

A radio-contrast agent may be administered via any suitable route. In certain embodiments, a radio-contrast agent may be administered parenterally, for example intravenously, subcutaneously, intravascularly, intramuscularly, intraperitoneal, intraabdominally, intraarterially, or intrathecally (the spine). In some embodiments, a radio-contrast agent may be administered in an amount effective for the purpose of providing a radiographical image. The effective amount may vary depending on factors such as the subject's weight or height, the subject's health and ability to clear the radio-contrast agent from the body, the route of administration, and other factors known to those of ordinary skill in the art.

According to aspects of the invention, a tissue protective agent is one that protects against radio-contrast induced kidney damage or radio-contrast nephropathy. A tissue protective agent may reduce or prevent radio-contrast induced kidney damage or radio-contrast nephropathy.

In certain embodiments, a tissue protective agent may be an agent that interacts with the erythropoietin receptor (Epo-R). In other embodiments, a tissue protective agent may be an agent that interacts with a receptor other than the Epo-R, or an agent that interacts with a receptor complex that may or may not include the Epo-R.

In some embodiments, a tissue protective agent that interacts with the Epo-R is an erythropoiesis stimulating agent. In some embodiments, the interaction of the erythropoiesis stimulating agent with the Epo-R activates the Epo-R. In certain embodiments, the interaction of an erythropoiesis-stimulating agent with the Epo-R stimulates erythropoiesis. An erythropoiesis-stimulating agent may bind to and activate the erythropoietin receptor on erythroid progenitor cells, and induce the proliferation and differentiation of erythroid progenitor cells into mature erythrocytes. Erythropoiesis stimulating agents include, but are not limited to, erythropoietin, (including different glycosylated forms of erythropoietin, epoetin-alpha, epoetin-beta, epoetin-delta and epoetin-omega), and darbepoetin (ARANESP™, novel erythropoiesis-stimulating protein (NESP), Amgen).

In some aspects of the invention, a tissue protective agent may interact with the Epo-R without inducing erythropoiesis. In certain embodiments, the interaction of the tissue protective agent with the Epo-R, without stimulating erythropoiesis, is sufficient to protect against kidney damage or injury. A tissue protective agent that does not stimulate erythropoiesis may interact with, for example, a heteroreceptor complex that includes the Epo-R and other receptors such as a β receptor subunit, for example CD131. Tissue protective agents that interact with alternative receptors include, but are not limited to, carbamylated erythropoietin (CEPO).

According to aspects of the invention, a tissue protective agent is administered to a subject in therapeutically effective amounts. Effective amounts are well known to those of ordinary skill in the art and are described in the literature. A therapeutically effective amount will be determined by the parameters discussed below; but, in any event, is that amount which establishes a level of a therapeutic or combination of therapeutics effective for treating a subject, such as a human subject, to prevent or reduce radio-contrast induced kidney damage or radio-contrast nephropathy. An effective amount means that amount alone or with multiple doses, necessary to delay the onset of, inhibit completely or lessen the progression of or halt altogether the onset or progression of the condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The doses of the tissue protective agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

According to aspects of the invention, a tissue protective agent is administered to a subject in an effective amount to prevent radio-contrast induced kidney damage or radio-contrast nephropathy. In some embodiments, radio-contrast induced kidney damage or radio-contrast nephropathy is reduced or prevented. In some embodiments where the tissue protective agent is an erythropoiesis stimulating agent that interacts with the Epo-R, an effective amount is an amount of an erythropoiesis stimulating agent that activates the Epo-R. In some embodiments, an amount effective of an erythropoiesis stimulating agent is an amount that stimulates erythropoiesis. The effective amount will vary depending on factors such as the subject's condition, the amount of radio-contrast agent being administered, the timing of administration of the tissue protective agent, the route of administration, and other factors known to those of ordinary skill in the art.

According to aspects of the invention, a tissue protective agent includes, but is not limited to, erythropoietin (including different glycosylated forms of erythropoietin, epoetin-alpha, epoetin-beta, epoetin-delta and epoetin-omega), darbepoetin (ARANESP™, novel erythropoiesis-stimulating protein (NESP), Amgen), asialoerythropoietin, carbamylated erythropoietin, continuous erythropoietin receptor activator (CERA™, Roche), HIP prolyl hydroxylase inhibitors, angelica sinensis polysaccharides, EPREX™ (a pharmaceutical drug used for the treatment of anemia, Johnson & Johnson), NeoRecormon (epoetin-beta, Roche), or Dynepo (prepared in human cell cultures by Shire Pharmaceuticals). In some aspects of the invention, a synthetic non-recombinant pegylated, peptidic erythropoiesis-stimulating agent such as HEMATIDE™ (Affymax, Inc.) may be used.

According to certain aspects of the invention, a tissue protective agent may be administered at a time close enough to the administration of a radio-contrast agent to provide the protective effects to prevent kidney damage or injury. In certain embodiments, a tissue protective agent may be administered prior to the administration of a radio-contrast agent. In some embodiments, a tissue protective agent may be administered 3 days, 2 days, 24 hours, 12 hours, 8 hours, 6 hours, 5 hours, 4 hours, 3 hours, 2 hours, 1 hour, 30 minutes, 15 minutes, 10 minutes, 5 minutes, 4 minutes, 3 minutes, 2 minutes, or 1 minute prior to the administration of a radio-contrast agent. In some embodiments, a tissue protective agent is administered within 24 hours of the administration of a radio-contrast agent. In some embodiments, a tissue protective agent may be administered immediately prior to the administration of a radio-contrast agent, that is less than 1 minute prior to the administration of a radio-contrast agent. In other embodiments, a tissue protective agent may be administered simultaneously with a radio-contrast agent. In certain embodiments, a tissue protective agent may be administered immediately after the administration of a radio-contrast agent, that is, less than 1 minute after the administration of a radio-contrast agent. In some embodiments, a tissue protective agent may be administered 1 minute, 2 minutes, 3 minutes, 4 minutes, 5 minutes, 10 minutes, 15 minutes, 30 minutes, 1 hour, 2 hours, 3, hours, 4 hours, hours, 6 hours, 8 hours, 12 hours, 24 hours, 2 days or 3 days after the administration of a radio-contrast agent. In some embodiments, the tissue protective agent is administered within 24 hours of administering the radio-contrast agent.

In certain aspects of the invention, administration of a tissue protective agent may need to be repeated. Any further required or necessary treatment may be determined by those of ordinary skill in the art and may vary depending on various factors such as the subject's health, extent of the damage or injury to the kidney and other relevant factors as known to those of ordinary skill in the art. In some embodiments, a second, third or fourth dose of a tissue protective agent may be required. In some embodiments, a second, third or fourth dose of a tissue protective agent may be administered within the same 24 hours or may be administered over a period of time. A period of time may be 2 days, 3 days, 4 days, 5 days, 6 days, 1 week, 2 weeks, 3 weeks, 1 month, 2 months, 3 months, 4 months, 5 months, 6 months, 1 year or longer. In certain embodiments, treatment with a tissue protective agent may continue indefinitely or as required. Continuous treatment with a tissue protective agent may begin at a high frequency and then reduce over time and according to need.

In some aspects of the invention, a tissue protective agent may be administered by any suitable route. A tissue protective agent may be administered parenterally, for example intravenously, subcutaneously, intra-arterially, intramuscularly, intraperitoneal, or intraabdominally.

In certain aspects of the invention, a tissue protective agent may be administered to a subject in need of treatment or to prevent kidney damage in the subject. A subject in need of treatment may be a subject that is about to undergo radiographical examination. A subject in need of treatment may be a subject at high risk such as a subject with chronic kidney disease or diabetes mellitus. A subject having or at risk of having kidney disease or diabetes mellitus may also be a subject that is about to undergo radiographical examination. In certain embodiments, a subject is a human.

According to aspects of the invention, a subject may be any mammal, for example, a human, a cat, a dog, a monkey, and a horse. In preferred embodiments, a subject is a human.

According to aspects of the invention, the terms “treating” and “treatment” include prophylaxis and therapy. When provided prophylactically, a treatment may be administered to a subject in advance of radio-contrast induced kidney injury or radio-contrast nephropathy (e.g., to a patient at risk of kidney injury), or upon the development of early signs of kidney injury in a patient after radio-contrast administration. A prophylactic treatment serves to prevent, delay, or reduce the rate of onset of kidney injury or the appearance of symptoms associated with kidney injury. A prophylactic treatment may reduce the incidence and accelerate the recovery of renal function, i.e. accelerate kidney healing). When provided therapeutically, a treatment may be administered at (or shortly after) the onset of the appearance of symptoms of radio-contrast induced actual kidney injury or radio-contrast nephropathy. Therapy may include preventing, slowing, or stopping radio-contrast induced kidney injury or radio-contrast nephropathy, or certain symptoms associated with kidney injury. In some embodiments, a treatment may serve to reduce the severity and duration of radio-contrast induced kidney injury or radio-contrast nephropathy, or symptoms thereof. In some embodiments, treating a subject may involve halting or slowing the progression of radio-contrast induced kidney injury or radio-contrast nephropathy, or of one or more symptoms associated with kidney injury. In some embodiments, treating a subject may involve preventing, delaying, or slowing the onset or progression of long-term symptoms associated with radio-contrast induced kidney injury or radio-contrast nephropathy.

In aspects of the invention, kidney damage or injury may be any type of damage caused to the kidney as a result of the administration of a radio-contrast agent. Kidney damage or injury may be severe, moderate or minor. In some embodiments, kidney damage or injury is an acute transient impairment of renal function, for example, radio-contrast nephropathy. In certain embodiments, the kidney damage or injury is enhanced due to a preexisting condition. In some embodiments, a preexisting condition may be diabetes mellitus, chronic kidney disease, intravascular volume depletion or other conditions that cause damage or injury to the kidney. In certain embodiments, the damage or injury affects the renal tubular epithelial cells. In some embodiments, kidney damage or injury directly affecting the renal tubular cells may be associated with the activation of caspase enzymes.

In aspects of the invention, radio-contrast induced kidney damage or radio-contrast nephropathy may be associated with apoptosis. In certain embodiments, diagnosis of cell injury or cell death may be associated with the activation of caspase enzymes, such as those involved in apoptosis signaling pathways, for example caspase 3 and caspase 9. In some embodiments, administration of a tissue protective agent as a protection or treatment against radio-contrast induced kidney damage or radio-contrast nephropathy, may reduce the level of caspase enzymes and as a result reduce apoptosis.

According to aspects of the invention, compositions for the treatment or prevention of kidney damage are provided. A composition, according to the invention, may be a tissue protective agent in an amount effective to prevent radiocontrast induced kidney damage, and an amount effective of a radio-contrast agent to provide an image in a radiographical examination.

According to aspects of the invention, a kit for the treatment or prevention of kidney damage is provided. A kit may include a package containing a tissue protective agent in an amount effective to prevent radio-contrast induced kidney damage and a radio-contrast agent in an amount effective to perform a radiographical examination. A tissue protective agent may be included as a separate individual preparation and a radio-contrast agent as a separate individual preparation. In some embodiments, a tissue protective agent and a radio-contrast agent may be combined as a single preparation. In certain embodiments, the preparations, as single individual preparations or as a single combined preparation, may be prepared as a pharmaceutical composition.

A pharmaceutical composition may include a tissue protective agent, and a radio-contrast agent, in combination with any standard physiologically and/or pharmaceutically acceptable carriers which are known in the art. In certain embodiments, the composition may be sterile. In some embodiments, the composition contains a therapeutically effective amount of a tissue protective agent, and a radio-contrast agent, in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

In aspects of the invention, a tissue protective agent preparation, a radio-contrast agent preparation, or combined preparation may be packaged according to conventional methods. In some embodiments, a tissue protective agent preparation, a radio-contrast agent preparation, or combined preparation may be provided as liquids. In other embodiments, a tissue protective agent preparation, a radio-contrast agent preparation, or combined preparation may be provided as freeze-dried preparations. Instructions relating to reconstituting the freeze-dried preparation may also be included in the kit. In some embodiments, a tissue protective agent preparation, a radio-contrast agent preparation, or combined preparation may be maintained at room temperature without any decrease in activity. In other embodiments, a tissue protective agent preparation, a radio-contrast agent preparation, or combined preparation may be maintained under fridge or freezer conditions if storage for a longer period of time is required. In certain embodiments, a tissue protective agent preparation and a radio-contrast agent preparation may require storage at different temperatures. In some embodiments, a tissue protective agent preparation and a radio-contrast agent preparation may be provided in different forms, for example one preparation may be a liquid and the other preparation may be a freeze-dried preparation.

In some embodiments, a kit may further include instructions for use in the package.

According to aspects of the invention, methods for screening the toxicity of a radio-contrast agent in the presence of a tissue protective agent in vitro are provided. A method for screening the toxicity of a radio-contrast agent may involve contacting a cell culture with a tissue protective agent and a radio-contrast agent, incubating the cell culture with the tissue protective agent and the radio-contrast agent and determining cell viability of the cell culture. In certain embodiments, cell viability may be determined using a colorimetric assay, for example a MTT [3-(4,5-dimethylthiazol-2-yl)-2,5-diphenyltetrazolium bromide] assay. The MTT assay is used to determine the ability of a mitochondrial dehydrogenase enzyme from viable cells to cleave the tetrazolium rings of the pale yellow MTT and form a dark blue formazan crystal.

In aspects of the invention, the cell culture may be human kidney cells. In some embodiments, the cells may be human renal tubular epithelial HK-2 cells.

In aspects of the invention, a tissue protective agent may be added to the cell culture immediately prior to the addition of a radio-contrast agent. In other embodiments, a tissue protective agent may be added simultaneously with a radio-contrast agent. In certain embodiments, a tissue protective agent may be added after a radio-contrast agent. In some embodiments, the cell culture may be incubated in the presence of a radio-contrast agent for a period of time before the addition of a tissue protective agent. In some embodiments, a tissue protective agent may be added 1 minute, 2 minutes, 3 minutes, 4 minutes, 5 minutes, 10 minutes, 15 minutes, 30 minutes, 1 hour, 2 hours, 3, hours, 4 hours, 5 hours, 6 hours, 8 hours, 12 hours or 24 hours after or prior to incubation with a radio-contrast agent.

### EXAMPLES

### Example 1

### Materials

The iodinated radiographic contrast agents used in this study, diatrizoate (Hypaque-76; 370 mg iodine/ml), iohexol (OMNIPAQUE™; 350 mg iodine/ml) and iodixanol (VISIPAQUE™; 320 mg iodine/ml), were purchased from Amersham Health, Princeton, N.J. Epoetin alfa (EPOGEN®) and darbepoetin alfa (ARANESP®) were purchased from Amgen, Thousand Oaks, Calif.

### Cell Culture

HK-2, a proximal tubular cell line derived from normal human kidney, and LLC-PK1, a proximal renal tubular cell line of porcine origin, were obtained from the American Type Culture Collection ATCC (Manassas, Va.). HK-2 cells were grown in DMEM media (GIBCO) supplemented with 10% FBS (Hyclone), 15 mM HEPES, 2 mM L-glutamine, 50 U/ml penicillin, 50 μg/ml streptomycin (GIBCO), 0.1 μg/ml hydrocortisone, 5 μg/ml insulin, and 5 μg/ml apotransferrin (Sigma, St. Louis, Mo.). LLC-PK1 cells were maintained in medium 199 (GIBCO) supplemented with 10% FBS and penicillin-streptomycin. For the planned experiments, cells were seeded on 24-well plates at a density of 10⁴ cells/cm² and cultured at 37° C. for 24 hours.

### Cell Viability

Cells were exposed to iodinated radiocontrast media (100 mg iodine/ml) for 6 hours at 37° C., then washed twice with Dulbecco PBS solution, and incubated in serum-free culture medium for 24 hours at 37° C. The cell viability was assessed by the mitochondrial activity in reducing MTT to form azan using a commercially available kit (Cell Proliferation Kit, Roche, Penzberg, Germany). In brief, cells were incubated at 37° C. for 4 hours in 200 μl of serum-free, phenol red free medium containing 20 μl of the MTT labeling reagent. 200 μl of solubilization solution were added to each well and cells were incubated at 37° C. for 24 hours. Aliquots of the incubation medium were transferred to a 96-well microplate and absorbance was measured at 570 nm with the reference of 690 nm using a microplate reader (MRX-II, Dynex).

### Measurement of Caspase-3 Activity

Activity of caspase-3 was measured by the degradation of subtype-specific peptide substrate after exposure of LLC-PK1 cells to iohexol or iodixanol. In brief, cells were seeded in 96-well plates and treated with radiocontrast media as described. After removal of the media, 100 μl of the Caspase-Glo 3/7 reagent (Promega, Madison, Wis.) were added to each well, and cells were incubated at room temperature for 1 hour. Luminescence of each sample was measured on a luminometer (Lumat LB9507, EGG Berthold). As a control for specificity, DEVD-CHO (Calbiochem, EMD Biosciences), a caspase-3 inhibitor, was added to appropriate wells during incubation in a final concentration of 50 μM. In parallel, cells were stained in wells with crystal violet and the absorption at 600 nm was measured. For each well, the luminometer data were normalized to the level of crystal violet absorption.

### Effects of a Variety of Radiocontrast Media on Cell Viability

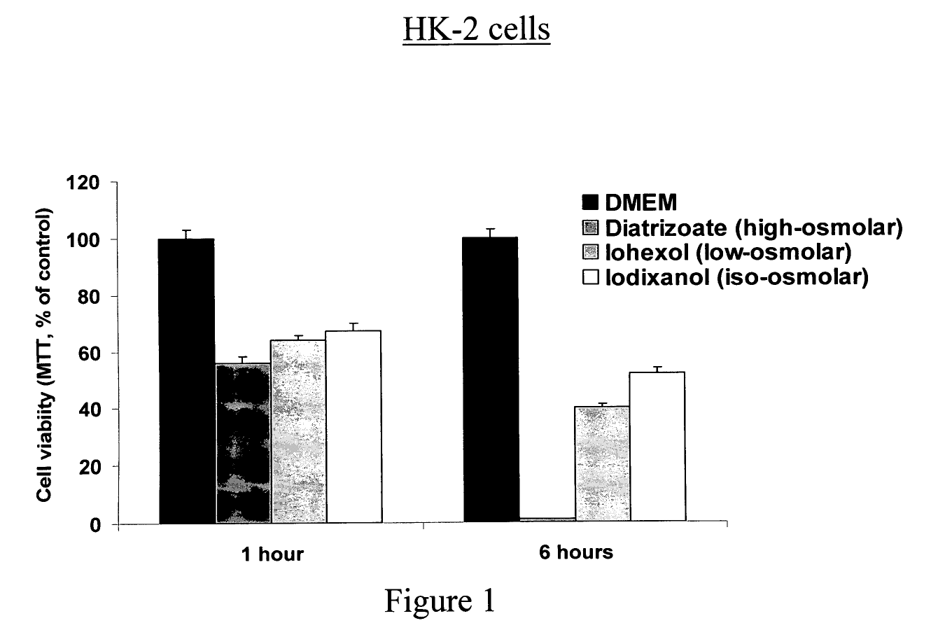

The effects of ionic and nonionic iodinated radiocontrast media on the viability of the human renal tubular epithelial cell line HK-2 were compared. As determined by the mitochondrial dehydrogenase ability to reduce MTT, radiocontrast media reduced the viability of cells exposed to 100 mg iodine/mL of contrast media for 1 or 6 hours (FIG. 1). The ionic high-osmolar radio-contrast agent diatrizoate (high-osmolar) reduced cell viability by 44% after 1-hour incubation and by 99% after a 6-hour incubation. The non-ionic radio-contrast agent iohexol (low-osmolar) reduced cell viability by 36% after 1-hour and by 60% after 6-hour incubation. The non-ionic radio-contrast agent iodixanol (iso-osmolar) reduced cell viability by 33% and 48% after 1-hour and 6-hour incubation, respectively.

Similar results were obtained with the porcine proximal tubular cell line LLC-PK1 (data not shown). Since iohexol and iodixanol, although designed to be non-toxic and currently widely used in the clinical setting, caused marked cell damage in HK-2 and LLC-PK1 cells, we used these two radio-contrast agents for the in vitro models aimed at examining whether ESAs have a protective effect.

Protective Effect of rhEpo Against Radiocontrast Induced Injury in Renal Tubular Epithelial Cells

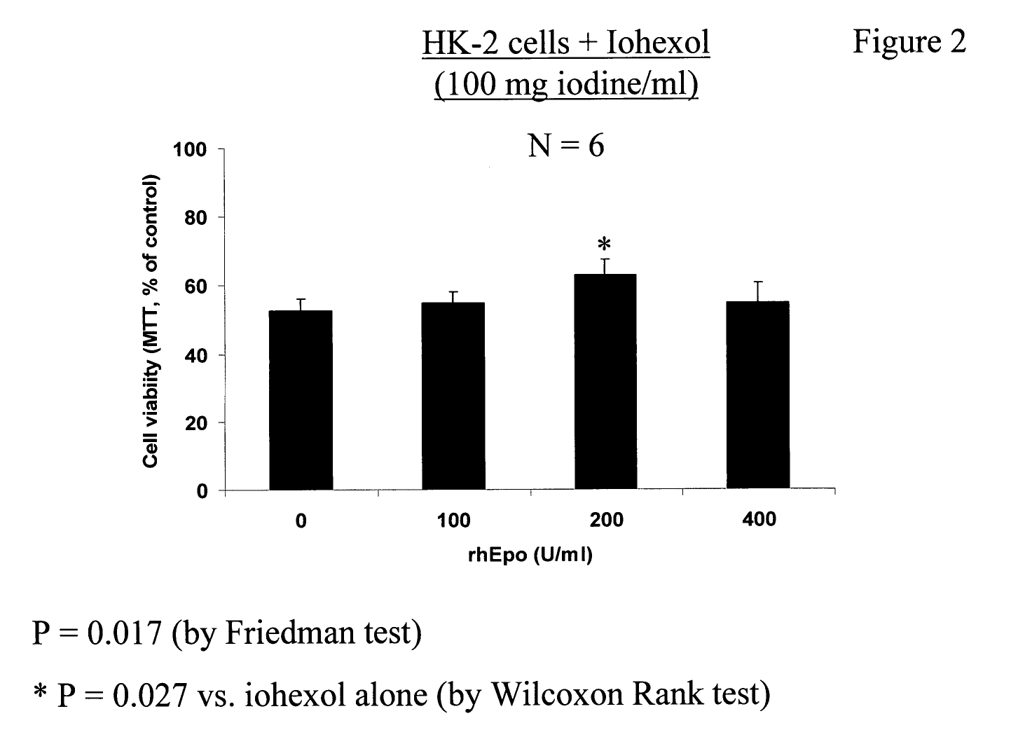

To investigate the potential protective effect of rhEpo on renal damage induced by radiocontrast media, we treated HK-2 cells for 6 hours with iohexol in the presence or absence of three different concentrations of rhEpo. As shown in FIG. 2, the cellular toxicity of iohexol on HK-2 cells was partially reversed by rhEpo, and this protective effect was dose dependent, with a ceiling effect provided by the 200 U/ml concentration of rhEpo.

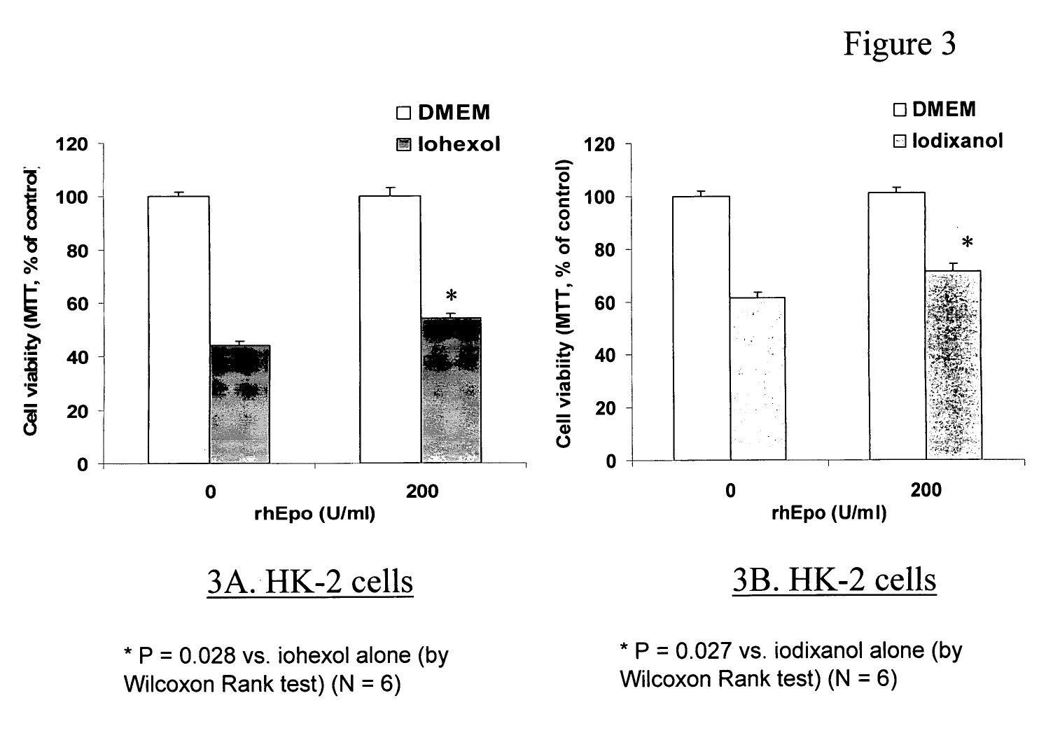

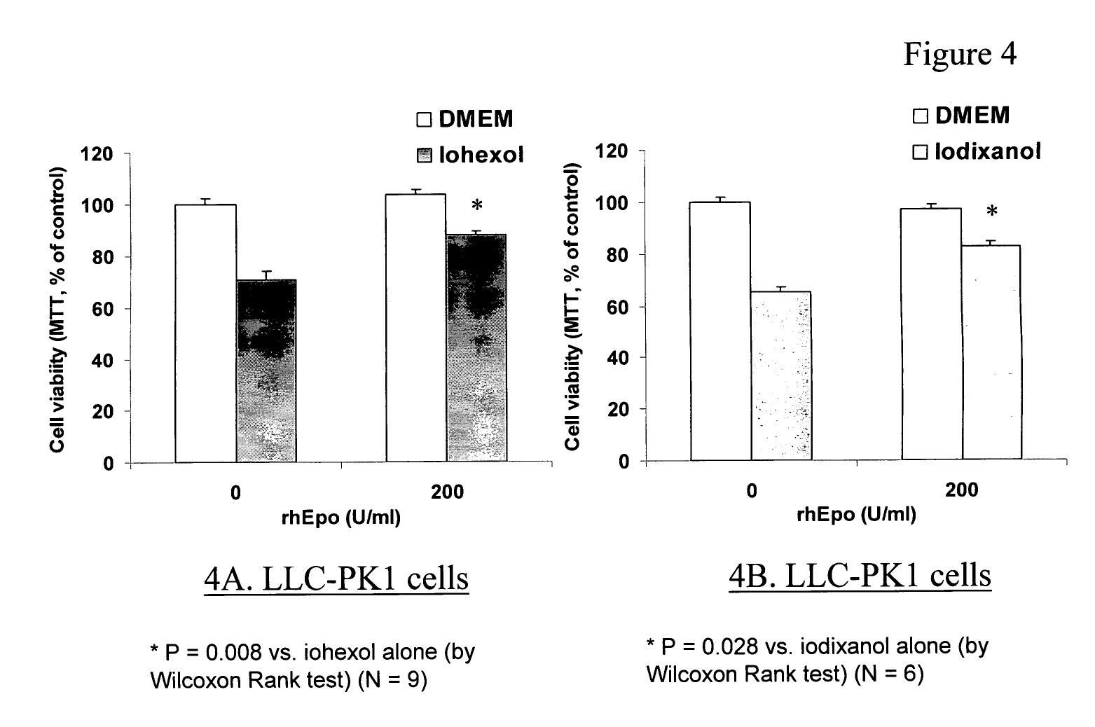

Treatment with 200 U/ml rhEpo was similarly protective against the cytotoxic effect of iodixanol on HK-2 cells (FIG. 3). Similar results were obtained for the porcine LLC-PK1 cells (FIG. 4).

### Protective Effect of Darbepoetin Against Radiocontrast Induced Injury in Renal Tubular Epithelial Cells

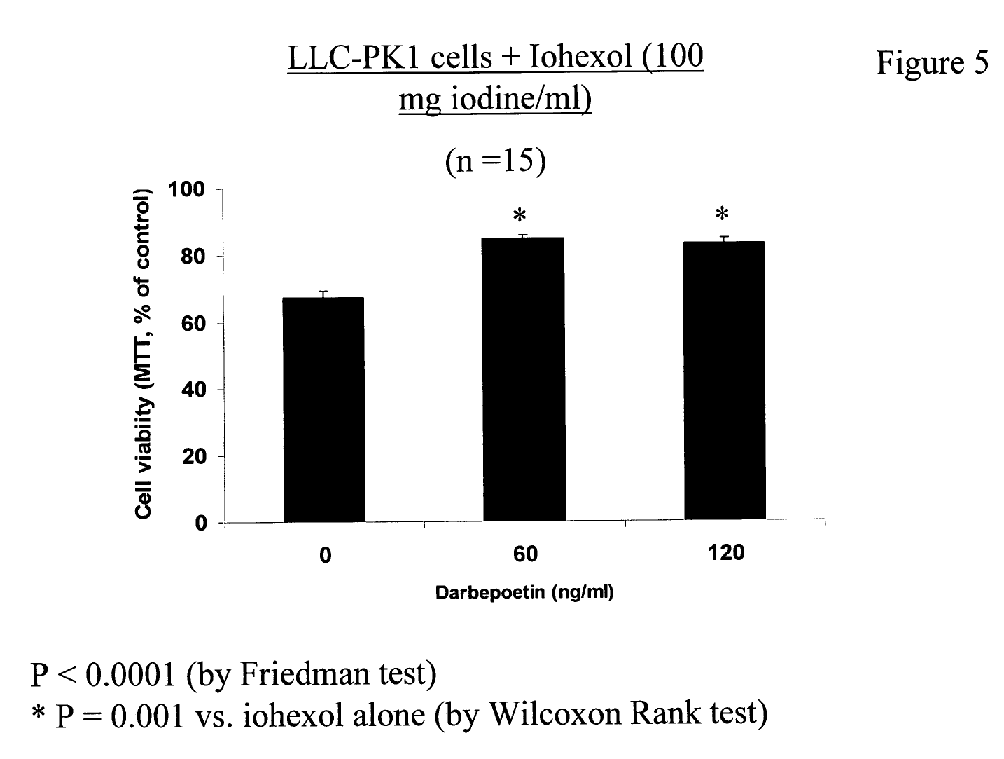

Co-treatment of LLC-PK1 cells with darbepoetin provided a similar level of protection against iohexol-induced injury, with a ceiling effect reached at a concentration of 60 ng/ml (FIG. 5).

### Caspase-3 Activation After Exposure of LLC-PK1 Cells to Radiocontrast Media

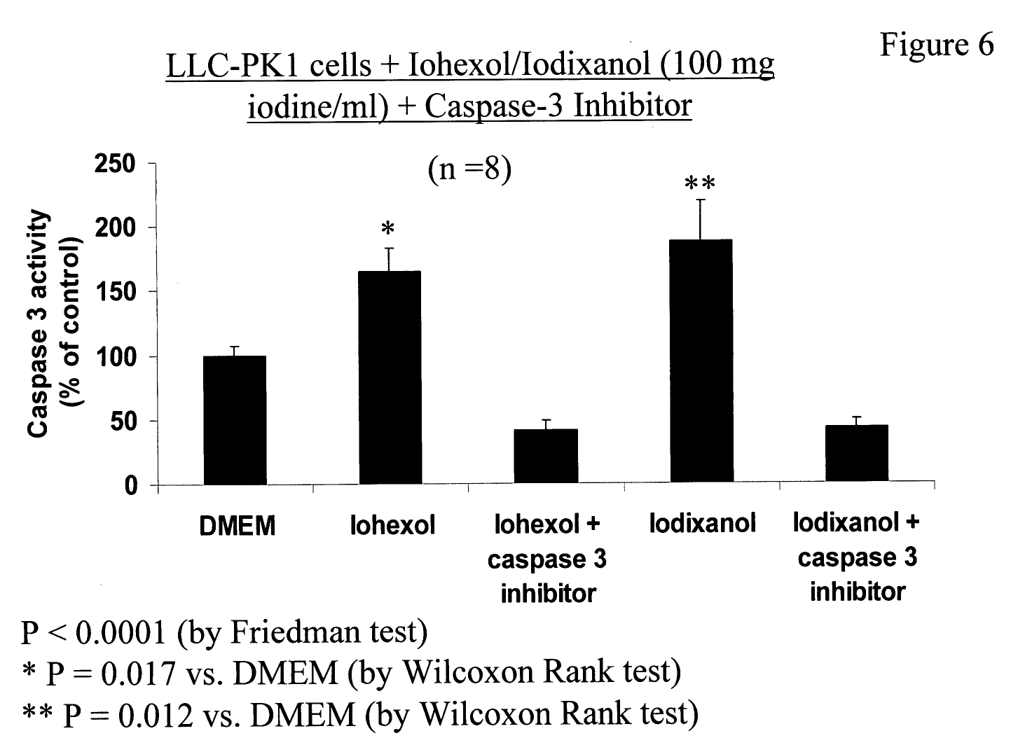

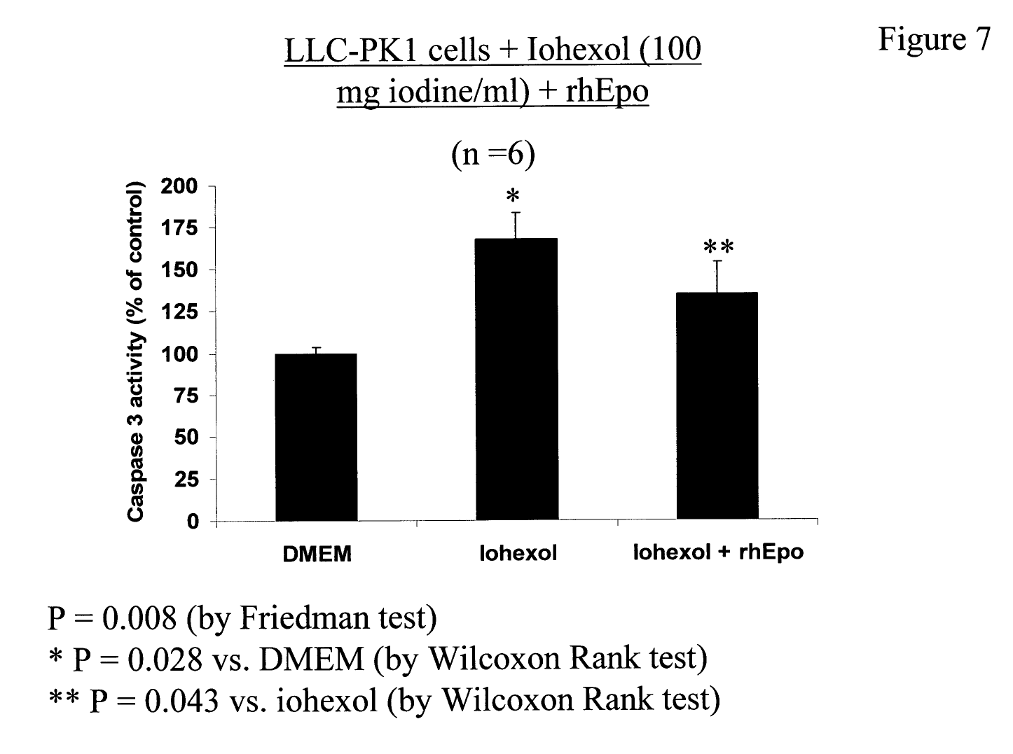

As shown in FIG. 6, iohexol and iodixanol induced an increase in caspase-3 activity, which was abrogated by co-treatment of cells with a caspase-3 inhibitor. Co-treatment of LLC-PK1 cells with rhEpo suppressed iohexol-induced caspase-3 activation (FIG. 7).

### Discussion

Radiocontrast nephropathy is a major complication that develops after radiographical examination with intravascular administration of iodinated contrast materials. Procedures that use iodinated radiographic contrast media, including cardiac angiography, are being used more frequently for both diagnostic and therapeutic purposes (Sheldon W C., Catheter Cardiovasc Interv, 2001, 53(1):40-5). In addition, two of the major risk factors for RCN, chronic kidney disease, and diabetes mellitus are also increasing in prevalence (Coresh J, et al., Am J Kidney Dis, 2003, 41(1):1-12; King H, et al., Diabetes Care, 1998, 21(9):1414-31). Both of these factors suggest that RCN will increase in incidence.

Although little is known about cellular mechanisms underlying RCN, direct toxic action on renal tubular epithelial cells is implicated in its pathogenesis (Humes H D, et al., Am J Physiol, 1987, 252(2 Pt 2):F246-55; Haller C, Hizoh I., Invest Radiol, 2004, 39(3):149-54). In our study we used the human renal tubular epithelial cell line HK-2 to determine the toxic effects of contrast media on renal tubular cells. We found that diatrizoate, iohexol and iodixanol had similar toxic effects after 1-hour incubation, but diatrizoate displayed more toxicity after 6-hour incubation. In healthy volunteers, radiocontrast medium is rapidly excreted (50% in 2 hours) by the kidneys almost exclusively through glomerular filtration (Lorusso V, et al. Invest Radiol, 2001, 36(6):309-16). However, the elimination half-life increases progressively with increasing renal impairment (Lorusso V, et al. Invest Radiol, 2001, 36(6):309-16), and for example, in patients with chronic renal failure, the elimination half-life of iopamidol is about 70 hours (Donnelly P K, et al., Invest Radiol, 1993, 28(7):629-32). Therefore, a 6-hour incubation used in our experimental model is clinically relevant.

Erythropoietin, the principal hematopoietic cytokine produced by the kidney and during prenatal development by the liver, regulates mammalian erythropoiesis and exhibits diverse cellular effects in non-hematopoietic tissues (Sasaki R., Intem Med, 2003, 42(2):142-9). The introduction of rhEpo has marked a significant advance in the management of anemia and unveiled its potential cardio- and neuro-protective actions (Sadamoto Y, et al., Biochem Biophys Res Commun, 1998, 253(1):26-32; Moon C, et al., J Pharmacol Exp Ther, 2006, 316(3):999-1005). We tested the ability of rhEpo to protect renal tubular epithelial cells from iodinated radiographic contrast media-induced toxicity. Using the MTT cell viability assay, we found that co-treatment with rhEpo reduced by 20% the toxicity of iohexol and iodixanol. Darbepoetin had a similar effect. We also demonstrated that the induction of caspase-3 activation by iohexol could be reduced by 20% by co-treatment of cells with rhEpo.

In conclusion, renal protection induced by rhEpo and its analogues against radiocontrast media can be demonstrated in an in vitro experimental cell culture model. Clinical trials are needed to assess the efficacy and safety of these drugs in humans.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method for treating or preventing in a subject kidney damage associated with the administration of a radio-contrast agent, comprising
administering to the subject a tissue protective agent in an amount effective to treat or prevent radio-contrast induced kidney damage or radio-contrast nephropathy, wherein the tissue protective agent is an erythropoiesis-stimulating agent; and
administering to the subject a radio-contrast agent in an amount effective to perform a radiographical examination.

2. (canceled)

3. The method of claim 1, wherein the erythropoiesis-stimulating agent is selected from the group consisting of erythropoietin, epoetin-alpha, epoetin-beta, epoetin-delta, epoetin-omega, darbepoetin, asialoerythropoietin, carbamylated erythropoietin, continuous erythropoietin receptor activator, HIF prolyl hydroxylase inhibitors, angelica sinensis polysaccharides, EPREX™, NeoRecormon (epoetin-beta), Dynepo, and a synthetic non-recombinant pegylated, peptidic erythropoiesis-stimulating agent.

4-6. (canceled)

7. The method of claim 1, wherein the tissue protective agent is darbepoietin.

8-9. (canceled)

10. The method of claim 1, wherein the radio-contrast agent is selected from the group consisting of Diatrizoate (amidotrizoic acid, or 3,5-Diacetamido-2,4,6-triiodobenzoic acid), Metrizoate (water-soluble, nephrotropic, high osmolar X-ray contrast media), Ioxaglate (water-soluble, nephrotropic, low osmolar X-ray contrast media), Iopamidol (water-soluble, nephrotropic, low osmolar X-ray contrast media), Iohexol, Iopromide (water-soluble, nephrotropic, low osmolar X-ray contrast media), and Iodixanol.

11-13. (canceled)

14. The method of claim 1, wherein the tissue protective agent is administered prior to the radio-contrast agent.

15. The method of claim 1, wherein the tissue protective agent is administered after the radio-contrast agent.

16. The method of claim 1, wherein the subject has chronic kidney disease.

17. The method of claim 1, wherein the subject has diabetes mellitus.

18. A composition for the treatment or prevention of kidney damage in a subject associated with the administration of a radio-contrast agent, comprising
a tissue protective agent in an amount effective to treat or prevent radio-contrast induced kidney damage or radio-contrast nephropathy, wherein the tissue protective agent is an erythropoiesis-stimulating agent, and
a radio-contrast agent in an amount effective to perform a radiographical examination.

19. (canceled)

20. The composition of claim 18, wherein the erythropoiesis-stimulating agent is selected from the group consisting of erythropoietin, epoetin-alpha, epoetin-beta, epoetin-delta, epoetin-omega, darbepoetin, asialoerythropoietin, carbamylated erythropoietin, continuous erythropoietin receptor activator, HIF prolyl hydroxylase inhibitors, angelica sinensis polysaccharides, EPREX™, NeoRecormon (epoetin-beta), Dynepo, and a synthetic non-recombinant pegylated, peptidic erythropoiesis-stimulating agent.

21-23. (canceled)

24. The composition of claim 18, wherein the tissue protective agent is darbopoietin.

25-26. (canceled)

27. The composition of claim 18, wherein the radio-contrast agent is selected from the group consisting of Diatrizoate (amidotrizoic acid, or 3,5-Diacetamido-2,4,6-triiodobenzoic acid), Metrizoate (water-soluble, nephrotropic, high osmolar X-ray contrast media), Ioxaglate (water-soluble, nephrotropic, low osmolar X-ray contrast media), Iopamidol (water-soluble, nephrotropic, low osmolar X-ray contrast media), Iohexol, Iopromide (water-soluble, nephrotropic, low osmolar X-ray contrast media), and Iodixanol.

28-29. (canceled)

30. A kit comprising
a package containing a tissue protective agent in an amount effective to treat or prevent radio-contrast induced kidney damage or radio-contrast nephropathy, wherein the tissue protective agent is an erythropoiesis-stimulating agent;
a radio-contrast agent an amount effective to perform a radiographical examination; and
instructions for use.

31. (canceled)

32. The kit of claim 30 wherein the erythropoiesis-stimulating agent is selected from the group consisting of erythropoietin, epoetin-alpha, epoetin-beta, epoetin-delta, epoetin-omega, darbepoetin, asialoerythropoietin, carbamylated erythropoietin, continuous erythropoietin receptor activator, HIF prolyl hydroxylase inhibitors, angelica sinensis polysaccharides, EPREX™, NeoRecormon (epoetin-beta), Dynepo, and a synthetic non-recombinant pegylated, peptidic erythropoiesis-stimulating agent.

33-35. (canceled)

36. The kit of claim 30, wherein the tissue protective agent is darbepoietin.

37-38. (canceled)

39. The kit of claim 30, wherein the radio-contrast agent is selected from the group consisting of Diatrizoate (amidotrizoic acid, or 3,5-Diacetamido-2,4,6-triiodobenzoic acid), Metrizoate (water-soluble, nephrotropic, high osmolar X-ray contrast media), Ioxaglate (water-soluble, nephrotropic, low osmolar X-ray contrast media), Iopamidol (water-soluble, nephrotropic, low osmolar X-ray contrast media), Iohexol, Iopromide (water-soluble, nephrotropic, low osmolar X-ray contrast media), and Iodixanol.

40-43. (canceled)

44. A method for screening a tissue protective agent in vitro, comprising
contacting a cell culture with a tissue protective agent and a radio-contrast agent;
incubating the cell culture with the tissue protective agent and the radio-contrast agent, wherein the tissue protective agent is an erythropoiesis-stimulating agent; and
determining cell viability of the cell culture.

45. (canceled)

46. The method of claim 44, wherein the erythropoiesis-stimulating agent is selected from the group consisting of erythropoietin, epoetin-alpha, epoetin-beta, epoetin-delta, epoetin-omega, darbepoetin, asialoerythropoietin, carbamylated erythropoietin, continuous erythropoietin receptor activator, HIF prolyl hydroxylase inhibitors, angelica sinensis polysaccharides, EPREX™, NeoRecormon (epoetin-beta), Dynepo, and a synthetic non-recombinant pegylated, peptidic erythropoiesis-stimulating agent.

47. The method of claim 44, wherein the cell culture comprises human kidney cells.

48-49. (canceled)

50. The method of claim 44, wherein the radio-contrast agent is selected from the group consisting of Diatrizoate (amidotrizoic acid, or 3,5-Diacetamido-2,4,6-triiodobenzoic acid), Metrizoate (water-soluble, nephrotropic, high osmolar X-ray contrast media), Ioxaglate (water-soluble, nephrotropic, low osmolar X-ray contrast media), Iopamidol (water-soluble, nephrotropic, low osmolar X-ray contrast media), Iohexol, Iopromide (water-soluble, nephrotropic, low osmolar X-ray contrast media), and Iodixanol.

51-55. (canceled)
